(19) United States
(12) Patent Application Publication
(10) Pub. No.: US 2025/0302532 A1
(43) Pub. Date: Oct. 2, 2025

(54) EVANESCENT OPTICAL FIBERS FOR LASER LITHOTRIPSY

(71) Applicant: Shockwave Medical, Inc., Santa Clara, CA (US)

(72) Inventors: Thomas Charles HASENBERG, Campbell, CA (US); Steven Yihlih PENG, Fremont, CA (US)

(73) Assignee: Shockwave Medical, Inc., Santa Clara, CA (US)

(21) Appl. No.: 18/622,587

(22) Filed: Mar. 29, 2024

(51) Int. Cl. A61B 18/26; A61B 18/00; A61B 18/22

(52) U.S. Cl. CPC A61B 18/26; A61B 2018/0022; A61B 2018/2222; A61B 2018/2244; A61B 2018/263

(57) ABSTRACT

A laser lithotripsy system may include a light energy source and a catheter. The catheter may comprise an optical fiber comprising a core and a cladding that encases the core. The optical fiber may be configured to be optically coupled to receive laser light from the light energy source. The optical fiber may include at least one evanescent portion and at least one non-evanescent portion. When light propagates through the core, an evanescent field may be transmitted out of the optical fiber in the evanescent portion and may not transmitted out of the optical fiber in the non-evanescent portion.

### FIELD

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. The energy from this electrical discharge enters the surrounding fluid faster than the speed of sound, generating an acoustic shock wave. In addition, the energy creates one or more rapidly expanding and collapsing vapor bubbles that generate secondary shock waves. The shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that generate shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with a fluid (using a relatively low pressure of 2-4 atm) so that the balloon expands to contact the lesion but is not an inflation pressure that substantively displaces the lesion. Energy can then be delivered to the catheter inside the balloon to produce acoustic shock waves that propagate through the walls of the angioplasty balloon and into the lesions, for example by applying voltage pulses across electrodes (if the energy source is electrohydraulic) or by transmitting a laser pulse into the fluid (if the energy source is a laser). Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure.

Known laser lithotripsy procedures transmit laser light into a fluid using end-firing optical fibers. However, end-firing optical fibers limit the directions in which laser light can be emitted. As a result, targeting certain obstructions—for instance, asymmetric obstructions or obstructions that are for the most part flush with a wall of a bodily structure—using conventional laser lithotripsy techniques can be challenging. Further, including light energy as a shock wave source in intravascular lithotripsy may be challenging because a single optical fiber generally emits light energy only at its distal tip; thus, to have multiple shock wave generating regions in a long angioplasty balloon, multiple optical fibers with multiple light emitting distal tips may be required, adding undesirable bulk to the catheter profile.

### SUMMARY

Provided are catheters for performing laser lithotripsy that utilize evanescent electromagnetic fields to produce shock waves for breaking down obstructions in bodily structures. A catheter may include an optical fiber, portions of which may be materially and geometrically configured to allow evanescent modes of propagating laser light to be transmitted out of the fiber and into a shock wave medium (e.g., a bodily fluid or a fluid contained in a balloon component of the catheter). Laser lithotripsy systems and methods for performing laser lithotripsy are also described.

The disclosed catheters may include optical fibers that vary geometrically along their length. The variations in the geometric dimensions of the fiber may create non-evanescent portions of the fiber in which evanescent fields decay (almost) entirely within the cladding and cannot be refracted into a medium outside of the optical fiber and evanescent portions of the fiber in which evanescent fields can be transmitted from the cladding into a medium outside of the optical fiber. The medium outside of the optical fiber may be a fluid, for example a fluid contained in a balloon carried by the catheter. Evanescent fields that are transmitted into the fluid from an evanescent portion of the fiber may produce bubbles that rapidly expand and collapse, generating shock waves. If the catheter is positioned in a bodily structure (e.g., a blood vessel) such that an evanescent portion of the optical fiber is in a vicinity of an obstruction (e.g., a calcified lesion in the vessel), these shock waves may be incident upon the obstruction, causing it to fragment.

A provided laser lithotripsy system may include a light energy source and a catheter comprising an optical fiber. The optical fiber may have a core and a cladding that encases the core and may be configured to be optically coupled to receive laser light from the light energy source. The optical fiber may include at least one evanescent portion and at least one non-evanescent portion. When light propagates through the core, an evanescent field may be transmitted out of the optical fiber in the evanescent portion and may not be not transmitted out of the optical fiber in the non-evanescent portion.

The core may have a first radius in the non-evanescent portion and a second radius less than the first radius in the evanescent portion. The first radius may be greater than or equal to 325 μm and less than or equal to 375 μm and the second radius may be greater than or equal to 50 μm and less than or equal to 350 μm. Starting at an interface between the non-evanescent portion and the evanescent portion, the core radius may taper from the first radius to the second radius. In some embodiments, the optical fiber includes a first non-evanescent portion and a second non-evanescent portion, and the evanescent portion may be between the first and second non-evanescent portions. The core may have the second radius in a central region of the evanescent portion. The core radius may taper from the first radius to the second radius in the evanescent portion between an interface between the first non-evanescent portion and the evanescent portion and the central region and may expand from the second radius to the first radius between the central region and an interface between the evanescent portion and the second non-evanescent portion. A taper angle of the core may be greater than or equal to 0.5° and less than or equal to 5°.

The cladding may have a first outer radius in the non-evanescent portion and a second outer radius less than the first outer radius in the evanescent portion. The first outer radius may be greater than or equal to 375 μm and less than or equal to 425 μm and the second outer radius may be greater than or equal to 50 μm and less than or equal to 375 μm. Starting at an interface between the non-evanescent portion and the evanescent portion, the cladding outer radius may taper from the first outer radius to the second outer radius. In some embodiments, the optical fiber includes a first non-evanescent portion and a second non-evanescent portion, wherein the evanescent portion is between the first and second non-evanescent portions. The cladding may have the second outer radius in a central region of the evanescent portion. The cladding outer radius may taper from the first thickness to the second outer radius in the evanescent portion between an interface between the first non-evanescent portion and the evanescent portion and the central region and from the second outer radius to the first outer radius between the central region and an interface between the evanescent portion and the second non-evanescent portion. A taper angle of the cladding may be greater than or equal to 0.5° and less than or equal to 5°. The cladding outer radius may taper continuously from the first outer radius to the second outer radius. Alternatively, the tapering cladding outer radius from the first outer radius to the second outer radius may be variable.

A ratio of a cladding outer radius in the non-evanescent portion to the cladding outer radius in the evanescent portion may be greater than 1 and less than or equal to 6.5. A ratio of a core radius in the non-evanescent portion to the core radius in the evanescent portion may be greater than 1 and less than or equal to 6.5. A ratio of a cladding outer radius to a core radius in the non-evanescent portion may be between 0.5 and 0.9. A ratio of a cladding outer radius to a core radius in the evanescent portion may be between 0.025 and 0.875. The cladding may be between 10% and 50% thinner in the evanescent portion than in the non-evanescent portion. A cross-sectional shape of the cladding and/or a cross-sectional shape of the core in the evanescent portion may be asymmetrical.

In some embodiments, at least 55% of the laser light received by the optical fiber is transmitted out of the evanescent portion. In some embodiments, the optical fiber comprises a first evanescent portion and a second evanescent portion, wherein greater than or equal to 35% and less than or equal to 40% of the laser light received by the optical fiber is transmitted out of the first evanescent portion and greater than or equal to 25% and less than or equal to 30% of the laser light received by the optical fiber is transmitted out of the second evanescent portion. In some embodiments, the optical fiber comprises a first evanescent portion, a second evanescent portion, and a third evanescent portion, wherein greater than or equal to 30% and less than or equal to 40% of the laser light received by the optical fiber is transmitted out of the first evanescent portion, greater than or equal to 15% and less than or equal to 20% of the laser light received by the optical fiber is transmitted out of the second evanescent portion, and greater than or equal to 15% and less than or equal to 20% of the laser light received by the optical fiber is transmitted out of the third evanescent portion.

A distal end of the optical fiber may be configured to emit light that is not transmitted out of the optical fiber at the evanescent portion. Greater than or equal to 25% and less than or equal to 40% of the laser light received by the optical fiber may be emitted at the distal end of the optical fiber.

The catheter may include a balloon. The optical fiber may be contained within the balloon. The balloon may be configured to contain a fluid. For a wavelength of the light energy source, the fluid may have an absorption coefficient of at least 100 cm⁻¹. The fluid may be an aqueous fluid. Alternatively, the catheter may include an enclosure and the optical fiber may be contained within the enclosure.

The light energy source may be a laser light source. A wavelength of the light energy source may be between 1 μm and 3 μm. For a wavelength of the light energy source, an index of refraction of the core may be greater than an index of refraction of the cladding. In some embodiments, for wavelength of the light energy source, an index of refraction of the core is between 1.43 and 1.44 and an index of refraction of the cladding is between 1.4 and 1.42. An optical power density of light emitted by the light energy source may be between 0.01 W/cm² and 1×10¹¹ W/cm².

### BRIEF DESCRIPTION OF THE FIGURES

The invention will now be described, by way of example only, with reference to the accompanying drawings, in which:

FIG. 1 shows a block diagram of a laser lithotripsy system, according to some embodiments.

FIG. 2 shows a cross-sectional side view of an optical fiber in a catheter that is configured for laser lithotripsy, according to some embodiments.

FIG. 3A shows the intensity of the electric field in the cladding and in the core of a non-evanescent portion of an optical fiber, according to some embodiments.

FIG. 3B shows the intensity of the electric field in the cladding and in the core of a non-evanescent portion of an optical fiber, according to some embodiments.

FIG. 4A shows simulated laser light in a catheter that is configured for laser lithotripsy, according to some embodiments.

FIG. 4B shows a close-up view of the simulated laser light shown in an evanescent portion of the catheter shown in FIG. 4A.

FIG. 5A shows a cross-sectional head-on view of an optical fiber that has a first geometric configuration, according to some embodiments.

FIG. 5B shows a cross-sectional head-on view of an optical fiber that has a second geometric configuration, according to some embodiments.

FIG. 5C shows a cross-sectional head-on view of an optical fiber that has a third geometric configuration, according to some embodiments.

FIG. 5D shows a cross-sectional head-on view of an optical fiber that has a fourth geometric configuration, according to some embodiments.

FIG. 6 shows a laser lithotripsy system with a balloon catheter, according to some embodiments.

FIG. 7 shows a laser lithotripsy system with an end-firing optical fiber, according to some embodiments.

FIG. 8A shows a block diagram of a laser lithotripsy system with one evanescent portion and an end-firing portion, according to some embodiments.

FIG. 8B shows a simulation of a laser lithotripsy system with one evanescent portion and an end-firing portion, according to some embodiments.

FIG. 8C shows simulated laser light in the laser lithotripsy system shown in FIG. 8B.

FIG. 9A shows a block diagram of a laser lithotripsy system with two evanescent portions and an end-firing portion, according to some embodiments.

FIG. 9B shows a simulation of a laser lithotripsy system with two evanescent portions and an end-firing portion, according to some embodiments.

FIG. 9C shows simulated laser light in the laser lithotripsy system shown in FIG. 9B.

FIG. 10A shows a block diagram of a laser lithotripsy system with three evanescent portions and an end-firing portion, according to some embodiments.

FIG. 10B shows a simulation of a laser lithotripsy system with three evanescent portions and an end-firing portion, according to some embodiments.

FIG. 10C shows simulated laser light in the laser lithotripsy system shown in FIG. 10B.

FIG. 11 shows a laser lithotripsy method, according to some embodiments.

FIG. 12 shows a computer system, according to some embodiments.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Disclosed are catheters for performing laser lithotripsy that leverage evanescent electromagnetic fields to produce shock waves for breaking down obstructions in bodily structures. A catheter may include an optical fiber, portions of which may be geometrically configured to allow evanescent modes of propagating laser light to be transmitted out of the fiber and into a shock wave medium (e.g., a bodily fluid or a fluid contained in a balloon component of the catheter). Laser lithotripsy systems and methods for performing laser lithotripsy are also provided.

Optical fibers such as those in the provided catheters may comprise a light-guiding core and a cladding that encases the core. The cladding may have a lower index of refraction than the core so that light that enters the fiber at a suitable angle experiences total internal reflection at the interface between the core and the cladding, thereby causing the light to be propagated along the length of the fiber. The angles at which the light can enter the fiber in order to be transmitted through the fiber may depend on the smallest angle of incidence at the core-cladding interface for which total internal reflection occurs (the so-called “critical angle”) for the index of refraction of the core, the index of refraction of the cladding, and the wavelength of the light. More specifically, light may be transmitted through the fiber provided that the light enters the fiber at an angle relative to the fiber's longitudinal axis such that the angle of incidence of the light at the core-cladding interface is greater than the critical angle.

The laws that govern the behavior of electromagnetic fields (described mathematically by Maxwell's equations) cause electromagnetic fields undergoing total internal reflection to partially penetrate into the external medium (e.g., the cladding) from the internal medium (e.g., the core). The penetrating field, known as the “evanescent” field, decays exponentially with increasing distance into the external medium. Under certain conditions (e.g., if the internal and external media have certain geometric properties), the evanescent fields can be refracted into medium outside of the optical fiber (that is, on the opposite side of the cladding as the core) that is different from the internal medium in which the electromagnetic field is reflected and the external medium into which the evanescent field penetrates.

A disclosed catheter may include an optical fiber that varies geometrically (and/or varies in its material composition) along its length. The variations in the geometric properties of the fiber may create non-evanescent portions of the fiber in which evanescent fields decay (almost) entirely within the cladding and cannot be refracted into a third medium and evanescent portions of the fiber in which evanescent fields can be transmitted (refracted) from the cladding into a third medium. As used herein, an evanescent field decaying entirely or almost entirely may refer to a field whose intensity decays by 99% or more, 99.9% or more, or 99.99% or more. The medium outside of the optical fiber may be a fluid (e.g., an aqueous fluid such as saline). In the evanescent portions of the fiber, a portion of the light (e.g., 10-50%) propagating through the fiber may leak out of the core, into the cladding, and into the fluid. Evanescent fields that are transmitted into the fluid from an evanescent portion of the fiber may heat the fluid and produce bubbles that rapidly expand and collapse, generating shock waves. If the catheter is positioned in a bodily structure (e.g., a blood vessel) such that an evanescent portion of the optical fiber is in a vicinity of an obstruction (e.g., a calcified lesion in the vessel), these shock waves may be incident upon the obstruction, causing it to fragment.

The catheters, systems, and methods described herein have several technical advantages. The optical fibers in the catheters can be manufactured using modified existing techniques, for example modified techniques for fabricating optical fibers for telecommunication or trace gas detectors. During fabrication, an optical fiber may be tailored to target specific types of obstructions (e.g., asymmetric lesions) by tuning the geometric properties of the emitting regions. A single catheter can comprise an optical fiber with multiple emitting regions, enabling lesions to be targeted at numerous locations and from numerous angles. Accordingly, the provided catheters, systems, and methods may substantially increase the efficiency and effectiveness of lithotripsy procedures.

Any of the systems, methods, techniques, and/or features disclosed herein may be combined, in whole or in part, with any other systems, methods, techniques, and/or features disclosed herein.

Reference to “about” or “approximately” a value or parameter herein includes (and describes) variations of that value or parameter per se. For example, description referring to “approximately X” or “about X” includes description of “X” as well as variations of “X”.

When a range of values or values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

“Bodily structures” can include any portion of any body part, for example any portion of a circulatory system, a urinary tract, or a digestive system.

“Evanescent fields” or “evanescent waves” refer to electromagnetic fields that penetrates into the cladding of an optical fiber when light in the core of the optical fiber undergoes total internal reflection at the interface of the core and the cladding. The terms “evanescent field” and “evanescent wave” may be used interchangeably.

The “critical angle” is the smallest angle of incidence at an interface between two media for which total internal reflection in one medium will occur.

Two objects are “geometrically similar” to one another the objects have the same shape. Geometrically similar shapes may have different sizes or orientations. If two shapes are geometrically similar, a first shape of the two shapes may be transformed into the second shape of the two shapes by uniformly scaling the first shape, translating the first shape in space, rotating the first shape, and/or reflecting the first shape over an axis.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

### Laser Lithotripsy System

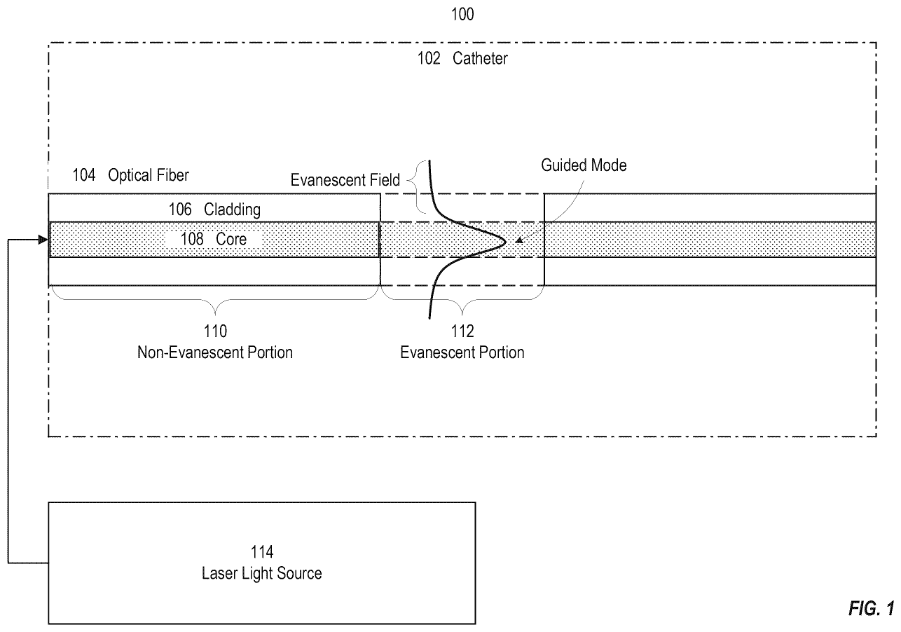

FIG. 1 shows a block diagram of an exemplary laser lithotripsy system 100. System 100 may include a catheter 102 that comprises an optical fiber 104. Optical fiber 104 may be formed from a core 108 and a cladding 106 that encases core 108 and may be optically coupled to receive laser light from a laser light source 114. System 100 may be configured for use in any lithotripsy procedure, including lithotripsy procedures for removing kidney stones, gallstones, bladder stones, ureter stones, urethral stones, bezoars, and calcified lesions in blood vessels.

Laser light source 114 may provide light to core 108 at an angle relative to the longitudinal axis of optical fiber 104 such that, when the light hits the interface between core 108 and cladding 106, the angle of incidence of the light on the interface is greater than or equal to the critical angle. That is, laser light source 114 may be configured to provide light to core 108 such that the light undergoes total internal reflection within core 108, which may cause the light to be transmitted along the length of optical fiber 104. In some embodiments, laser light source 114 may be replaced with another suitable light energy source.

Optical fiber 104 may include at least one non-evanescent portion 110 and at least one evanescent portion 112. When the light that is provided to optical fiber 104 by laser light source 114 undergoes total internal reflection, the reflected electromagnetic field may partially penetrate into cladding 106 as an evanescent field. In non-evanescent portion 110, core 108 and cladding 106 may be configured such that the penetrating evanescent field is constrained to (i.e., is not transmitted out of) optical fiber 104. That is, the evanescent field resulting from light in core 108 undergoing total internal reflection in non-evanescent portion 110 may be negligible or non-existent outside of optical fiber 104. In evanescent portion 112, on the other hand, core 108 and cladding 106 may be configured such that the evanescent field is transmitted out of optical fiber 104 and into a surrounding medium.

Core 108 and cladding 106 may comprise the same materials or may have different material compositions. In some embodiments, both core 108 and cladding 106 comprise silica glass.

The index of refraction of cladding 106 may be greater than 2, between 1 and 2, between 1 and 1.9, between 1 and 1.8, between 1 and 1.7, between 1 and 1.6, or between 1 and 1.5. For example, the index of refraction of cladding 106 may be approximately 1.4, 1.41, 1.42, 1.43, or 1.44. In some embodiments, the index of refraction of cladding 106 is between 1.4 and 1.42, for example approximately 1.41, 1.411, 1.412, 1.413, 1.414, 1.415, 1.416, 1.417, 1.418, or 1.419. In some embodiments, the index of refraction of cladding 106 is between 1.419 and 1.42, for example approximately 1.4191, 1.4192, 1.4193, 1.4194, 1.4195, 1.4196, 1.4197, 1.4198, or 1.4199. Cladding 106 may have a lower index of refraction (for a given wavelength of light) than core 108.

The index of refraction of core 108 may be greater than 2, between 1 and 2, between 1 and 1.9, between 1 and 1.8, between 1 and 1.7, between 1 and 1.6, or between 1 and 1.5. For example, the index of refraction of core 108 may be approximately 1.4, 1.41, 1.42, 1.43, or 1.44. In some embodiments, the index of refraction of core 108 is between 1.43 and 1.44, for example approximately 1.431, 1.432, 1.433, 1.434, 1.435, 1.436, 1.437, 1.438, or 1.439. In some embodiments, the index of refraction for core 108 is between 1.436 and 1.437, for example approximately 1.4361, 1.4362, 1.4363, 1.4364, 1.4365. 1.4367, 1.4368, or 1.4369. Core 108 may have a higher index of refraction (for a given wavelength of light) than cladding 106.

Laser light source 114 may comprise any suitable laser, for example a Tm:YAG laser, InGaAs diode laser (980 nm), a Nd:YAG laser, a pulsed dye laser, a holmium YAG laser, or a thulium fiber laser. The light received by optical fiber 104 from laser light source 114 may have a wavelength between 1 μm and 3 μm, between 1.25 μm and 2.75 μm, between 1.5 μm and 2.5 μm, or between 1.75 μm and 2.25 μm. In some embodiments, the received by optical fiber 104 from laser light source 104 has a wavelength less than 1 μm or greater than 3 μm. In some embodiments, the received by optical fiber 104 from laser light source 104 has a wavelength of approximately 1.5 μm, 1.55 μm, 1.6 μm, 1.65 μm, 1.7 μm, 1.8 μm, 1.85 μm, 1.9 μm, 1.95 μm, 2 μm, 2.05 μm, 2.1 μm, 2.15 μm, 2.2 μm, 2.25 μm, 2.3 μm, 2.35 μm, 2.4 μm, 2.45 μm, or 2.5 μm. In some embodiments, the received by optical fiber 104 from laser light source 104 has a wavelength within 20%, 15%, 12%, 10%, 8%, 6%, 5%, 4%, 3%, 2%, 1%, 0.75%, 0.5%, 0.25% or 0.1% of 1.5 μm, 1.55 μm, 1.6 μm, 1.65 μm, 1.7 μm, 1.8 μm, 1.85 μm, 1.9 μm, 1.95 μm, 2 μm, 2.05 μm, 2.1 μm, 2.15 μm, 2.2 μm, 2.25 μm, 2.3 μm, 2.35 μm, 2.4 μm, 2.45 μm, or 2.5 μm.

Laser light source 114 may have an optical power between 0.1 W and 500 W, between 1 W and 450 W, between 1 W and 400 W, between 1 W and 350 W, between 1 W and 300 W, between 1 W and 250 W, between 1 W and 200 W, between 1 W and 150 W, or between 1 W and 50 W. In some embodiments, laser light source 114 has an optical power less than or equal to 1 W or greater than or equal to 500 W. In some embodiments, laser light source 114 has an optical power of approximately 1 W, 2 W, 3 W, 4 W, 5 W, 10 W, 20 W, 25 W, 50 W, 75 W, 100 W, 125 W, 150 W, 175 W, or 200 W. In some embodiments, laser light source 114 has an optical power within 20%, 15%, 12%, 10%, 8%, 6%, 5%, 4%, 3%, 2%, 1%, 0.75%, 0.5%, 0.25% or 0.1% of 1 W, 2 W, 5 W, 10 W, 20 W, 25 W, 50 W, 75 W, 100 W, 125 W, 150 W, 175 W, or 200 W.

Laser light source 114 may deliver pulses of light that have a power sufficient to induce generation of shock waves in aqueous media when emitted through either an evanescent portion or a distal end of an optical fiber. Advantageously, the power density of the emitted light may be low enough to not generate plasma within the aqueous media; generating plasma within such a confined volume may damage the catheter and/or injure tissue. In some embodiments, the emitted light from the optical fiber has a power density less than 1×10¹² W/cm². In some embodiments, the emitted light from the optical fiber has a power density between 0.01 W/cm²-1×10¹¹ W/cm². For example, the emitted light from the optical fiber may have a power density between 40 and 4×10⁵ W/cm². In other embodiments, the emitted light from the optical fiber has a power density between 1 kW/cm²-10 MW/cm².

Laser light source 114 may emit pulses of light. In some embodiments, the light pulses are emitted at a frequency between 1 Hz and 1 GHz, between 10 Hz and 1 MHz, or 100 Hz and 1 kHz. In some embodiments, the light pulses are emitted at a frequency less than 1 Hz or greater than 1 GHz. In some embodiments, the light pulses are emitted at a frequency of approximately 1 Hz, 50 Hz, 100 Hz, 150 Hz, 200 Hz, 250 Hz, 300 Hz, 350 Hz, 400 Hz, 450 Hz, 500 Hz, 550 Hz, 600 Hz, 650 Hz, 700 Hz, 750 Hz, 800 Hz, 900 Hz, 950 Hz, 1 kHz, 50 kHz, 100 kHz, 150 kHz, 200 kHz, 250 kHz, 300 kHz, 350 kHz, 400 kHz, 450 kHz, 500 kHz, 550 kHz, 600 kHz, 650 kHz, 700 kHz, 750 kHz, 800 kHz, 900 kHz, 950 kHz, 1 MHz, 50 MHz, 100 MHz, 150 MHz, 200 MHz, 250 MHz, 300 MHz, 350 MHz, 400 MHz, 450 MHz, 500 MHz, 550 MHz, 600 MHz, 650 MHz, 700 MHz, 750 MHz, 800 MHz, 900 MHz, 950 MHz, or 1 GHz. In some embodiments, the light pulses are emitted at a frequency within 20%, 15%, 12%, 10%, 8%, 6%, 5%, 4%, 3%, 2%, 1%, 0.75%, 0.5%, 0.25% or 0.1% of 1 Hz, 50 Hz, 100 Hz, 150 Hz, 200 Hz, 250 Hz, 300 Hz, 350 Hz, 400 Hz, 450 Hz, 500 Hz, 550 Hz, 600 Hz, 650 Hz, 700 Hz, 750 Hz, 800 Hz, 900 Hz, 950 Hz, 1 kHz, 50 kHz, 100 kHz, 150 kHz, 200 kHz, 250 kHz, 300 kHz, 350 kHz, 400 kHz, 450 kHz, 500 kHz, 550 kHz, 600 kHz, 650 kHz, 700 kHz, 750 kHz, 800 kHz, 900 kHz, 950 kHz, 1 MHz, 50 MHz, 100 MHz, 150 MHz, 200 MHz, 250 MHz, 300 MHz, 350 MHz, 400 MHz, 450 MHz, 500 MHz, 550 MHz, 600 MHz, 650 MHz, 700 MHz, 750 MHz, 800 MHz, 900 MHz, 950 MHz, or 1 GHz.

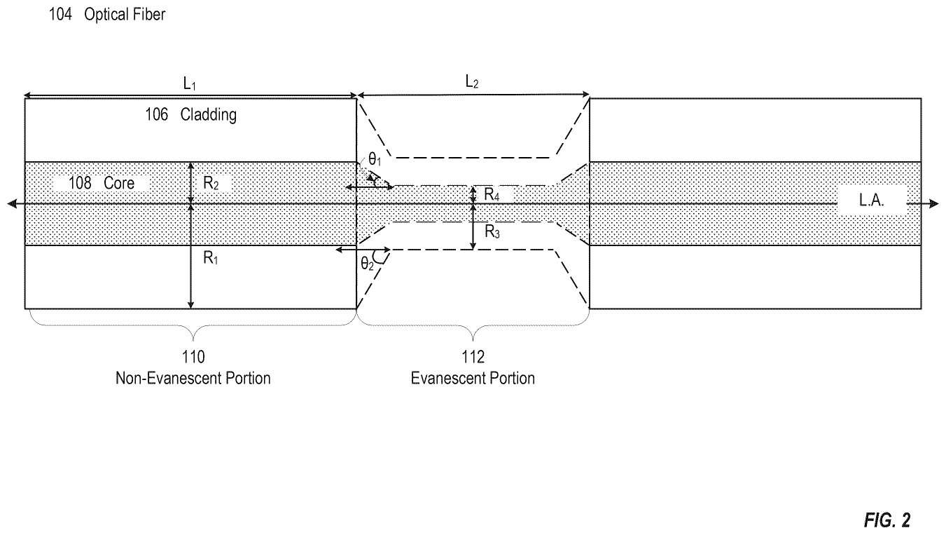

A cross-sectional view of an exemplary embodiment of optical fiber 104 is shown in FIG. 2. In non-evanescent portion 110, cladding 106 may have an outer radius R₁ (as measured from a central longitudinal axis of optical fiber 104, indicated by line L.A. in FIG. 2) and core may have a radius R₂ (as measured from the central longitudinal axis of optical fiber 104, indicated by line L.A. in FIG. 2). Starting at the interface between non-evanescent portion 110 and evanescent portion 112, the outer radius of cladding 106 may taper from an outer radius R₁ to an outer radius R₃<R₁. Similarly, starting at the interface between non-evanescent portion 110 and evanescent portion 112, the radius of core 108 may taper from radius R₂ to a radius R₄<R₂. If, as depicted in FIG. 2, evanescent portion 112 is sandwiched between two non-evanescent portions 110, the outer radius of cladding 106 may reach outer radius R₃ at a central region of evanescent portion 112 and then expand back to outer radius R₁ between the central region and the interface between evanescent portion 112 and the second non-evanescent portion 110. Likewise, if evanescent portion 112 is sandwiched between two non-evanescent portions 110, the radius of core may reach radius R₄ at a central region of evanescent portion 112 and then expand back to radius R₂ between the central region and the interface between evanescent portion 112 and the second non-evanescent portion 110.

The outer radius R₁ of cladding 106 in non-evanescent portion 110 may be greater than or equal to the radius R₂ of core 108 in non-evanescent portion 110. In some embodiments, R₁ is between 300 μm and 500 μm, between 325 μm and 475 μm, between 350 μm and 450 μm, or between 375 μm and 425 μm. In some embodiments, R₁ is less than 300 μm or greater than 500 μm. In some embodiments, R₁ is approximately 350 μm, 360 μm, 370 μm, 380 μm, 390 μm, 400 μm, 410 μm, 420 μm, 430 μm, 440 μm, 450 μm, 460 μm, 470 μm, 480 μm, 490 μm, or 500 μm. In some embodiments, R₁ is about 365 μm, 370 μm, 375 μm, 380 μm, 381 μm, 382 μm, 383 μm, 384 μm, 385 μm, 386 μm, or 387 μm. In some embodiments, R₁ is within 20%, 15%, 12%, 10%, 8%, 6%, 5%, 4%, 3%, 2%, 1%, 0.75%, 0.5%, 0.25% or 0.1% of 383 μm, 384 μm, 385 μm, or 386 μm.

The radius R₂ of core 108 in non-evanescent portion 110 may be between 250 μm and 450 μm, between 275 μm and 425 μm, between 300 μm and 400 μm, or between 325 μm and 375 μm. In some embodiments, R₂ is less than 250 μm or greater than 450 μm. In some embodiments, R₂ is approximately 300 μm, 310 μm, 320 μm, 340 μm, 350 μm, 360 μm, 370 μm, 380 μm, 390 μm, 400 μm, 410 μm, 420 μm, 430 μm, 440 μm, or 450 μm. In some embodiments, R₂ is about 345 μm, 346 μm, 347 μm, 348 μm, 349 μm, 350 μm, 351 μm, 352 μm, 353 μm, 354 μm, or 355 μm. In some embodiments, R₂ is within 20%, 15%, 12%, 10%, 8%, 6%, 5%, 4%, 3%, 2%, 1%, 0.75%, 0.5%, 0.25% or 0.1% of 348 μm, 349 μm, 350 μm, 351 μm, or 352 μm.

In non-evanescent portion 110, cladding 106 may have a first cladding radial thickness t₁, where t₁=R₁-R₂. The first cladding radial thickness t₁ may be between 50 μm and 250 μm, between 75 μm and 200 μm, between 100 μm and 175 μm, or between 125 μm and 150 μm. In some embodiments, the first cladding radial thickness t₁ is greater than 250 μm or less than 50 μm. In some embodiments, the first cladding radial thickness t₁ is approximately 50 μm, 60 μm, 70 μm, 80 μm, 90 μm, 100 μm, 110 μm, 120 μm, 130 μm, 140 μm, 150 μm, 160 μm, 170 μm, 180 μm 190 μm, or 200 μm.

The outer radius R₃ to which cladding 106 tapers in evanescent portion 112 may be between 10 μm and 400 μm, between 20 μm and 390 μm, between 30 μm and 380 μm, between 40 μm and 370 μm, between 45 μm and 365 μm, between 50 μm and 360 μm, or between 55 μm and 358 μm. In some embodiments, R₃ is less than 10 μm or greater than 400 μm. In some embodiments, R₃ is approximately 35 μm, 55 μm, 75 μm, 95 μm, 115 μm, 135 μm, 155 μm, 175 μm, 195 μm, 215 μm, 235 μm, 255 μm, 275 μm, 295 μm, 315 μm, 335 μm, 355 μm, or 375 μm. In some embodiments, R₃ is within 20%, 15%, 12%, 10%, 8%, 6%, 5%, 4%, 3%, 2%, 1%, 0.75%, 0.5%, 0.25% or 0.1% of 50 μm, 55 μm, 60 μm, 80 μm, 100 μm, 200 μm, 300 μm, 350 μm, or 360 μm.

The radius R₄ to which core 108 tapers in evanescent portion 112 may be between 10 μm and 350 μm, between 20 μm and 345 μm, between 30 μm and 340 μm, between 40 μm and 335 μm, between 45 μm and 330 μm, or between 50 μm and 325 μm. In some embodiments, R₄ is less than 10 μm or greater than 350 μm. In some embodiments, R₄ is approximately 25 μm, 50 μm, 75 μm, 100 μm, 125 μm, 150 μm, 175 μm, 200 μm, 225 μm, 250 μm, 275 μm, 300 μm, 325 μm, or 350 μm. In some embodiments, R₄ is within 20%, 15%, 12%, 10%, 8%, 6%, 5%, 4%, 3%, 2%, 1%, 0.75%, 0.5%, 0.25% or 0.1% of 50 μm, 100 μm, 150 μm, 200 μm, 250 μm, 300 μm, or 350 μm.

In evanescent portion 112, cladding 106 may have a second cladding radial thickness t₂, where t₂=R₃-R₄. The second cladding radial thickness t₂ may be between 10 μm and 390 μm, between 20 μm and 350 μm, between 30 μm and 300 μm, between 40 μm and 250 μm, or between 50 μm and 200 μm. In some embodiments, the second cladding radial thickness t₂ is greater than 390 μm or less than 10 μm. In some embodiments, the second cladding radial thickness t₂ is approximately 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, or 85 μm.

The ratio of the cladding outer radius R₁ in non-evanescent portion 110 to the cladding outer radius R₃ in evanescent portion 112 may be between 1 and 10, between 1 and 9, between 1 and 8, between 1 and 7, between 1 and 6, between 1 and 5, between 1 and 4, between 1 and 3, or between 1 and 2. In some embodiments, the ratio of R₁ to R₃ is less than 1 or greater than 10. In some embodiments, the ratio of R₁ to R₃ is approximately 1, 1.04, 1.08, 1.12, 1.16, 1.2, 1.24, 1.28, 1.32, 1.36, 1.4, 1.44, 1.48, 1.52, 1.56, 1.6, 1.64, 1.68, 1.72, 1.76, 1.8, 1.84, 1.88, 1.92, 1.96, 2, 2.04, 2.08, 2.12, 2.16, 2.2, 2.24, 2.28, 2.32, 2.36, 2.4, 2.44, 2.48, 2.52, 2.56, 2.6, 2.64, 2.68, 2.72, 2.76, 2.8, 2.84, 2.88, 2.92, 2.96, 3, 3.04, 3.08, 3.12, 3.16, 3.2, 3.24, 3.28, 3.32, 3.36, 3.4, 3.44, 3.48, 3.52, 3.56, 3.6, 3.64, 3.68, 3.72, 3.76, 3.8, 3.84, 3.88, 3.92, 3.96, 4, 4.04, 4.08, 4.12, 4.16, 4.2, 4.24, 4.28, 4.32, 4.36, 4.4, 4.44, 4.48, 4.52, 4.56, 4.6, 4.64, 4.68, 4.72, 4.76, 4.8, 4.84, 4.88, 4.92, 4.96, 5, 5.04, 5.08, 5.12, 5.16, 5.2, 5.24, 5.28, 5.32, 5.36, 5.4, 5.44, 5.48, 5.52, 5.56, 5.6, 5.64, 5.68, 5.72, 5.76, 5.8, 5.84, 5.88, 5.92, 5.96, 6, 6.04, 6.08, 6.12, 6.16, 6.2, 6.24, 6.28, 6.32, 6.36, 6.4, 6.44, 6.48, 6.52, 6.56, 6.6, 6.64, 6.68, 6.72, 6.76, 6.8, 6.84, 6.88, 6.92, 6.96, or 7. In some embodiments, the ratio of R₁ to R₃ is within 20%, 15%, 12%, 10%, 8%, 6%, 5%, 4%, 3%, 2%, 1%, 0.75%, 0.5%, 0.25% or 0.1% of 1.08, 1.12, 1.16, 1.2, 1.24, 1.28, 1.32, 1.36, 1.4, 1.44, 1.48, 1.52, 1.56, 1.6, 1.64, 1.68, 1.72, 1.76, 1.8, 1.84, 1.88, 1.92, 1.96, 2, 2.04, 2.08, 2.12, 2.16, 2.2, 2.24, 2.28, 2.32, 2.36, 2.4, 2.44, 2.48, 2.52, 2.56, 2.6, 2.64, 2.68, 2.72, 2.76, 2.8, 2.84, 2.88, 2.92, 2.96, 3, 3.04, 3.08, 3.12, 3.16, 3.2, 3.24, 3.28, 3.32, 3.36, 3.4, 3.44, 3.48, 3.52, 3.56, 3.6, 3.64, 3.68, 3.72, 3.76, 3.8, 3.84, 3.88, 3.92, 3.96, 4, 4.04, 4.08, 4.12, 4.16, 4.2, 4.24, 4.28, 4.32, 4.36, 4.4, 4.44, 4.48, 4.52, 4.56, 4.6, 4.64, 4.68, 4.72, 4.76, 4.8, 4.84, 4.88, 4.92, 4.96, 5, 5.04, 5.08, 5.12, 5.16, 5.2, 5.24, 5.28, 5.32, 5.36, 5.4, 5.44, 5.48, 5.52, 5.56, 5.6, 5.64, 5.68, 5.72, 5.76, 5.8, 5.84, 5.88, 5.92, 5.96, 6, 6.04, 6.08, 6.12, 6.16, 6.2, 6.24, 6.28, 6.32, or 6.36.

The ratio of the core radius R₂ in non-evanescent portion 110 to the core radius R₄ in evanescent portion 112 may be between 1 and 10, between 1 and 9, between 1 and 8, between 1 and 7, between 1 and 6, between 1 and 5, between 1 and 4, between 1 and 3, or between 1 and 2. In some embodiments, the ratio of R₂ to R₄ is less than 1 or greater than 10. In some embodiments, the ratio of R₂ to R₄ is approximately 1, 1.04, 1.08, 1.12, 1.16, 1.2, 1.24, 1.28, 1.32, 1.36, 1.4, 1.44, 1.48, 1.52, 1.56, 1.6, 1.64, 1.68, 1.72, 1.76, 1.8, 1.84, 1.88, 1.92, 1.96, 2, 2.04, 2.08, 2.12, 2.16, 2.2, 2.24, 2.28, 2.32, 2.36, 2.4, 2.44, 2.48, 2.52, 2.56, 2.6, 2.64, 2.68, 2.72, 2.76, 2.8, 2.84, 2.88, 2.92, 2.96, 3, 3.04, 3.08, 3.12, 3.16, 3.2, 3.24, 3.28, 3.32, 3.36, 3.4, 3.44, 3.48, 3.52, 3.56, 3.6, 3.64, 3.68, 3.72, 3.76, 3.8, 3.84, 3.88, 3.92, 3.96, 4, 4.04, 4.08, 4.12, 4.16, 4.2, 4.24, 4.28, 4.32, 4.36, 4.4, 4.44, 4.48, 4.52, 4.56, 4.6, 4.64, 4.68, 4.72, 4.76, 4.8, 4.84, 4.88, 4.92, 4.96, 5, 5.04, 5.08, 5.12, 5.16, 5.2, 5.24, 5.28, 5.32, 5.36, 5.4, 5.44, 5.48, 5.52, 5.56, 5.6, 5.64, 5.68, 5.72, 5.76, 5.8, 5.84, 5.88, 5.92, 5.96, 6, 6.04, 6.08, 6.12, 6.16, 6.2, 6.24, 6.28, 6.32, 6.36, 6.4, 6.44, 6.48, 6.52, 6.56, 6.6, 6.64, 6.68, 6.72, 6.76, 6.8, 6.84, 6.88, 6.92, 6.96, or 7. In some embodiments, the ratio of R₂ to R₄ is within 20%, 15%, 12%, 10%, 8%, 6%, 5%, 4%, 3%, 2%, 1%, 0.75%, 0.5%, 0.25% or 0.1% of 1.08, 1.12, 1.16, 1.2, 1.24, 1.28, 1.32, 1.36, 1.4, 1.44, 1.48, 1.52, 1.56, 1.6, 1.64, 1.68, 1.72, 1.76, 1.8, 1.84, 1.88, 1.92, 1.96, 2, 2.04, 2.08, 2.12, 2.16, 2.2, 2.24, 2.28, 2.32, 2.36, 2.4, 2.44, 2.48, 2.52, 2.56, 2.6, 2.64, 2.68, 2.72, 2.76, 2.8, 2.84, 2.88, 2.92, 2.96, 3, 3.04, 3.08, 3.12, 3.16, 3.2, 3.24, 3.28, 3.32, 3.36, 3.4, 3.44, 3.48, 3.52, 3.56, 3.6, 3.64, 3.68, 3.72, 3.76, 3.8, 3.84, 3.88, 3.92, 3.96, 4, 4.04, 4.08, 4.12, 4.16, 4.2, 4.24, 4.28, 4.32, 4.36, 4.4, 4.44, 4.48, 4.52, 4.56, 4.6, 4.64, 4.68, 4.72, 4.76, 4.8, 4.84, 4.88, 4.92, 4.96, 5, 5.04, 5.08, 5.12, 5.16, 5.2, 5.24, 5.28, 5.32, 5.36, 5.4, 5.44, 5.48, 5.52, 5.56, 5.6, 5.64, 5.68, 5.72, 5.76, 5.8, 5.84, 5.88, 5.92, 5.96, 6, 6.04, 6.08, 6.12, 6.16, 6.2, 6.24, 6.28, 6.32, or 6.36. The ratio of R₂ to R₄ may be (approximately) the same as or may be different than the ratio of R₁ to R₃.

Non-evanescent portion 110 may have a length L₁. Length L₁ may be between 0.5 mm and 40 mm, between 1 mm and 39 mm, between 2 mm and 38 mm, between 3 mm and 37 mm, between 4 mm and 36 mm, or between 5 mm and 35 mm. In some embodiments, L₁ is less than 0.5 mm or greater than 35 mm. In some embodiments, L₁ is approximately 1 mm, 5 mm, 10 mm, 15 mm, 20 mm, 25 mm, 30 mm, 35 mm, 40 mm, 45 mm, or 50 mm. In some embodiments, L₁ is within 20%, 15%, 12%, 10%, 8%, 6%, 5%, 4%, 3%, 2%, 1%, 0.75%, 0.5%, 0.25% or 0.1% of 1 mm, 5 mm, 10 mm, 15 mm, 20 mm, 25 mm, 30 mm, 35 mm, 40 mm, 45 mm, or 50 mm.

Evanescent portion 112 may have a length L₂. Length L₂ may be between 0.25 mm and 35 mm, between 0.5 mm and 30 mm, between 1 mm and 29 mm, between 2 mm and 28 mm, between 3 mm and 27 mm, or between 4 mm and 26 mm, 5 mm and 25 mm, 6 mm and 24 mm, 7 mm and 23 mm, 8 mm and 22 mm, 9 mm and 21 mm, or 10 mm and 20 mm. In some embodiments, L₂ is less than 0.25 mm or greater than 35 mm. In some embodiments, L₂ is approximately 1 mm, 2.5 mm, 5 mm, 7.5 mm, 10 mm, 12.5 mm, 15 mm, 17.25 mm, or 20 mm. In some embodiments, L₂ is within 20%, 15%, 12%, 10%, 8%, 6%, 5%, 4%, 3%, 2%, 1%, 0.75%, 0.5%, 0.25% or 0.1% of 1 mm, 2.5 mm, 5 mm, 7.5 mm, 10 mm, 12.5 mm, 15 mm, 17.25 mm, or 20 mm.

The ratio of core radius R₂ in non-evanescent portion 110 to cladding outer radius R₁ in non-evanescent portion 110 may be between 0.5 and 0.9, for example approximately 0.55, 0.6, 0.65, 0.7, 0.75, 0.8, or 0.85. In some embodiments, ratio of core radius R₂ in non-evanescent portion 110 to cladding outer radius R₁ in non-evanescent portion 110 is less than 0.5 or greater than 0.9. The ratio of core radius R₄ in evanescent portion 112 to cladding outer radius R₃ in evanescent portion 112 may be between 0.025 and 0.875, for example approximately 0.05, 0.075, 0.1, 0.125, 0.15, 0.175, 0.2, 0.225, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, or 0.85. In some embodiments, the ratio of core radius R₄ in evanescent portion 112 to cladding outer radius R₃ in evanescent portion 112 is greater than 0.875 or less than 0.025.

The tapering of cladding 106 between the interface of non-evanescent portion 110 and evanescent portion 112 and the region of evanescent portion 112 where cladding 106 has outer radius R₃ may be linear, multi-step, or curved. If the tapering of cladding 106 is curved, the curve may have a cross-sectional shape that is characterized by a quadratic, cubic, quartic, or other polynomial function, or a combination of polynomial and/or multi-step functions. Similarly, the tapering of core 108 between the interface of non-evanescent portion 110 and evanescent portion 112 and the region of evanescent portion 112 where core 108 has radius R₄ may be linear, multi-step, or curved. If the tapering of core 108 is curved, the curve may have a cross-sectional shape that is characterized by a quadratic, cubic, quartic, or other polynomial function, or a combination of polynomial and/or multi-step functions.

Core 108 may have a taper angle θ₁ in evanescent portion 112, as shown in FIG. 2. θ₁ may be between 0.05° and 10°, 0.1° and 9°, 0.2° and 8°, 0.3° and 7°, 0.4° and 6°, or 0.5° and 5°. In some embodiments, θ₁ may be less than 0.05° or greater than 9°. In some embodiments, θ₁ may be approximately 0.1°, 0.15°, 0.2°, 0.25°, 0.3°, 0.35°, 0.4°, 0.45°, 0.5°, 0.55°, 0.6°, 0.65°, 0.7°, 0.75°, 0.8°, 0.85°, 0.9°, 1°, 1.05°, 1.1°, 1.15°, 1.2°, 1.25°, 1.3°, 1.35°, 1.4°, 1.45°, 1.5°, 1.55°, 1.6°, 1.75°, 1.8°, 1.85°, 1.9°, 1.95°, 2°, 2.05°, 2.1°, 2.15°, 2.2°, 2.25°, 2.3°, 2.35°, 2.4°, 2.45°, 2.5°, 2.55°, 2.6°, 2.75°, 2.8°, 2.85°, 2.9°, 2.95°, 3°, 3.05°, 3.1°, 3.15°, 3.2°, 3.25°, 3.3°, 3.35°, 3.4°, 3.45°, 3.5°, 3.55°, 3.6°, 3.75°, 3.8°, 3.85°, 3.9°, 3.95°, 4°, 4.05°, 4.1°, 4.15°, 4.2°, 4.25°, 4.3°, 4.35°, 4.4°, 4.45°, 4.5°, 4.55°, 4.6°, 4.75°, 4.8°, 4.85°, 4.9°, 4.95°, 5°, 5.05°, 5.1°, 5.15°, 5.2°, 5.25°, 5.3°, 5.35°, 5.4°, 5.45°, 5.5°, 5.55°, 5.6°, 5.65°, 5.7°, 5.75°, 5.8°, 5.85°, 5.9°, 6°, 6.15°, 6.2°, 6.25°, 6.3°, 6.35°, 6.4°, 6.45°, 6.5°, 6.55°, 6.6°, 6.65°, 6.7°, 6.75°, 6.8°, 6.85°, 6.9°, 7°, 7.15°, 7.2°, 7.25°, 7.3°, 7.35°, 7.4°, 7.45°, 7.5°, 7.55°, 7.6°, 7.65°, 7.7°, 7.75°, 7.8°, 7.85°, 7.9°, 8°, 8.15°, 8.2°, 8.25°, 8.3°, 8.35°, 8.4°, 8.45°, 8.5°, 8.55°, 8.6°, 8.65°, 8.7°, 8.75°, 8.8°, 8.85°, 8.9°, 9°, 9.15°, 9.2°, 9.25°, 9.3°, 9.35°, 9.4°, 9.45°, 9.5°, 9.55°, 9.6°, 9.65°, 9.7°, 9.75°, 9.8°, 9.85°, or 0.9°. In some embodiments, θ₁ may be within 20%, 15%, 12%, 10%, 8%, 6%, 5%, 4%, 3%, 2%, 1%, 0.75%, 0.5%, 0.25% or 0.1%. of 0.1°, 0.15°, 0.2°, 0.25°, 0.3°, 0.35°, 0.4°, 0.45°, 0.5°, 0.55°, 0.6°, 0.65°, 0.7°, 0.75°, 0.8°, 0.85°, 0.9°, 1°, 1.05°, 1.1°, 1.15°, 1.2°, 1.25°, 1.3°, 1.35°, 1.4°, 1.45°, 1.5°, 1.55°, 1.6°, 1.75°, 1.8°, 1.85°, 1.9°, 1.95°, 2°, 2.05°, 2.1°, 2.15°, 2.2°, 2.25°, 2.3°, 2.35°, 2.4°, 2.45°, 2.5°, 2.55°, 2.6°, 2.75°, 2.8°, 2.85°, 2.9°, 2.95°, 3°, 3.05°, 3.1°, 3.15°, 3.2°, 3.25°, 3.3°, 3.35°, 3.4°, 3.45°, 3.5°, 3.55°, 3.6°, 3.75°, 3.8°, 3.85°, 3.9°, 3.95°, 4°, 4.05°, 4.1°, 4.15°, 4.2°, 4.25°, 4.3°, 4.35°, 4.4°, 4.45°, 4.5°, 4.55°, 4.6°, 4.75°, 4.8°, 4.85°, 4.9°, 4.95°, 5°, 5.05°, 5.1°, 5.15°, 5.2°, 5.25°, 5.3°, 5.35°, 5.4°, 5.45°, 5.5°, 5.55°, 5.6°, 5.65°, 5.7°, 5.75°, 5.8°, 5.85°, 5.9°, 6°, 6.15°, 6.2°, 6.25°, 6.3°, 6.35°, 6.4°, 6.45°, 6.5°, 6.55°, 6.6°, 6.65°, 6.7°, 6.75°, 6.8°, 6.85°, 6.9°, 7°, 7.15°, 7.2°, 7.25°, 7.3°, 7.35°, 7.4°, 7.45°, 7.5°, 7.55°, 7.6°, 7.65°, 7.7°, 7.75°, 7.8°, 7.85°, 7.9°, 8°, 8.15°, 8.2°, 8.25°, 8.3°, 8.35°, 8.4°, 8.45°, 8.5°, 8.55°, 8.6°, 8.65°, 8.7°, 8.75°, 8.8°, 8.85°, 8.9°, 9°, 9.15°, 9.2°, 9.25°, 9.3°, 9.35°, 9.4°, 9.45°, 9.5°, 9.55°, 9.6°, 9.65°, 9.7°, 9.75°, 9.8°, 9.85°, or 0.9°.

Cladding 106 may have a taper angle θ₂ in evanescent portion 112, as shown in FIG. 2. θ₂ may be between 0.05° and 10°, 0.1° and 9°, 0.2° and 8°, 0.3° and 7°, 0.4° and 6°, or 0.5° and 5°. In some embodiments, θ₂ may be less than 0.05° or greater than 9°. In some embodiments, θ₂ may be approximately 0.25°, 0.3°, 0.35°, 0.4°, 0.45°, 0.5°, 0.55°, 0.6°, 0.65°, 0.7°, 0.75°, 0.8°, 0.85°, 0.9°, 1°, 1.05°, 1.1°, 1.15°, 1.2°, 1.25°, 1.3°, 1.35°, 1.4°, 1.45°, 1.5°, 1.55°, 1.6°, 1.75°, 1.8°, 1.85°, 1.9°, 1.95°, 2°, 2.05°, 2.1°, 2.15°, 2.2°, 2.25°, 2.3°, 2.35°, 2.4°, 2.45°, 2.5°, 2.55°, 2.6°, 2.75°, 2.8°, 2.85°, 2.9°, 2.95°, 3°, 3.05°, 3.1°, 3.15°, 3.2°, 3.25°, 3.3°, 3.35°, 3.4°, 3.45°, 3.5°, 3.55°, 3.6°, 3.75°, 3.8°, 3.85°, 3.9°, 3.95°, 4°, 4.05°, 4.1°, 4.15°, 4.2°, 4.25°, 4.3°, 4.35°, 4.4°, 4.45°, 4.5°, 4.55°, 4.6°, 4.75°, 4.8°, 4.85°, 4.9°, 4.95°, 5°, 5.05°, 5.1°, 5.15°, 5.2°, 5.25°, 5.3°, 5.35°, 5.4°, 5.45°, or 5.5°. In some embodiments, θ₂ may be within 20%, 15%, 12%, 10%, 8%, 6%, 5%, 4%, 3%, 2%, 1%, 0.75%, 0.5%, 0.25% or 0.1%. of 0.25°, 0.3°, 0.35°, 0.4°, 0.45°, 0.5°, 0.55°, 0.6°, 0.65°, 0.7°, 0.75°, 0.8°, 0.85°, 0.9°, 1°, 1.05°, 1.1°, 1.15°, 1.2°, 1.25°, 1.3°, 1.35°, 1.4°, 1.45°, 1.5°, 1.55°, 1.6°, 1.75°, 1.8°, 1.85°, 1.9°, 1.95°, 2°, 2.05°, 2.1°, 2.15°, 2.2°, 2.25°, 2.3°, 2.35°, 2.4°, 2.45°, 2.5°, 2.55°, 2.6°, 2.75°, 2.8°, 2.85°, 2.9°, 2.95°, 3°, 3.05°, 3.1°, 3.15°, 3.2°, 3.25°, 3.3°, 3.35°, 3.4°, 3.45°, 3.5°, 3.55°, 3.6°, 3.75°, 3.8°, 3.85°, 3.9°, 3.95°, 4°, 4.05°, 4.1°, 4.15°, 4.2°, 4.25°, 4.3°, 4.35°, 4.4°, 4.45°, 4.5°, 4.55°, 4.6°, 4.75°, 4.8°, 4.85°, 4.9°, 4.95°, 5°, 5.05°, 5.1°, 5.15°, 5.2°, 5.25°, 5.3°, 5.35°, 5.4°, 5.45°, or 5.5°. Taper angle θ₂ may greater than, less than, or equal to taper angle θ₁.

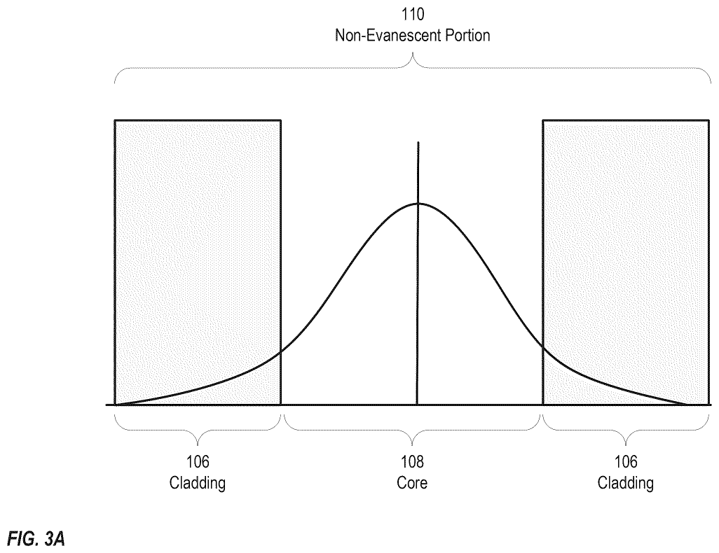

FIGS. 3A-3B illustrate the intensity of the electric field in a cross-section of an exemplary optical fiber in the non-evanescent portion 110 of the fiber (FIG. 3A) and in the evanescent portion 112 of the fiber (FIG. 3B). As shown, in both non-evanescent portion 110 and evanescent portion 112, the electric field propagating through core 108 may penetrate into cladding 106 when the electric field reflects off of the interface between core 106 and cladding 108. In non-evanescent portion 110 (FIG. 3A), the geometric properties of cladding 106 and core 108 of the optical fiber (e.g., the radii of cladding 106 and core 108, the cross-sectional shapes of cladding 106 and core 106, etc.) may be such that the electric field outside of cladding 106 is negligible or nonexistent. That is, outside cladding 106 in of non-evanescent portion 110, the amplitude of any evanescent field may be insufficient for the evanescent field to be transmitted into a medium surrounding the optical fiber. In evanescent portion 112 (FIG. 3B), on the other hand, the geometric properties of cladding 106 and core 108 of the optical fiber (e.g., the radii of cladding 106 and core 108, the cross-sectional shapes of cladding 106 and core 106, etc.) may be such that the electric field outside of cladding 106 is not negligible, i.e., that the amplitude of the evanescent field is sufficient for the evanescent field to be transmitted into a medium surrounding the optical fiber.

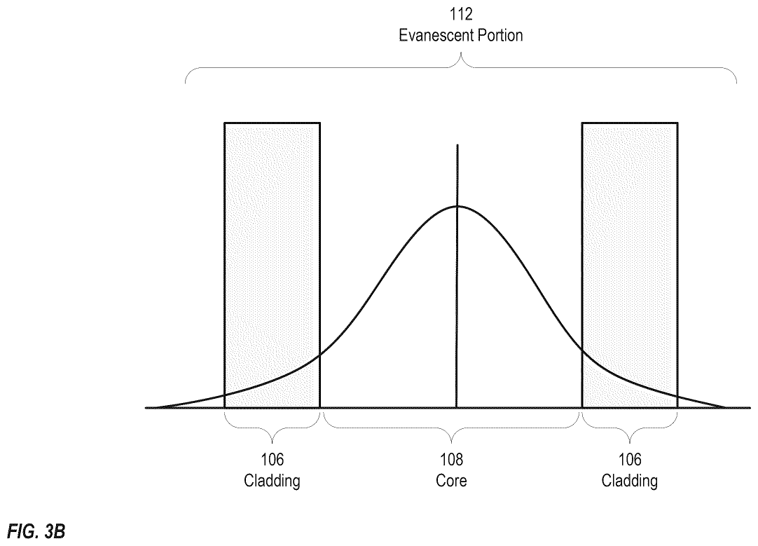

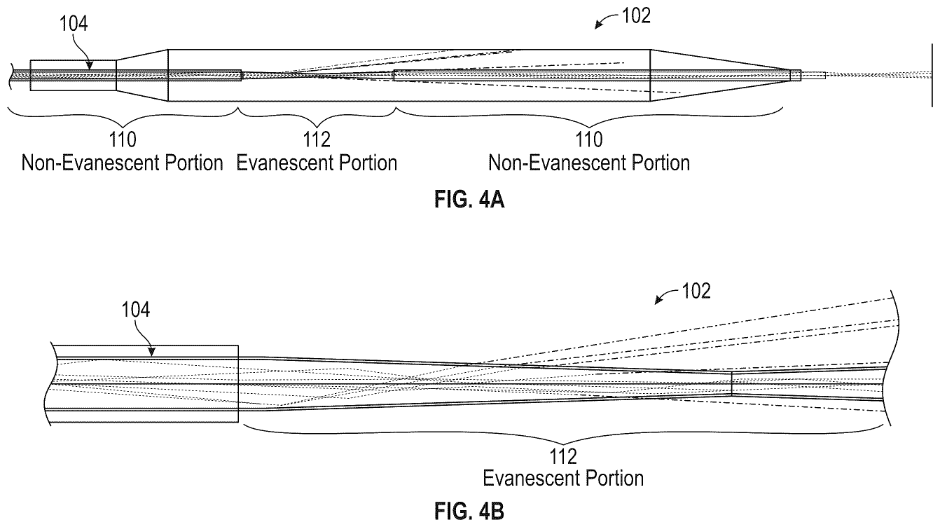

FIGS. 4A-4B show simulated laser light in an example catheter 102. In non-evanescent portions 110 of optical fiber 104 in catheter 102, the laser light—including the evanescent fields resulting from the light undergoing total internal reflection in the core of optical fiber 104 is contained within optical fiber—is contained within optical fiber. However, in evanescent portion 112 of optical fiber 104, the evanescent fields may be transmitted out of optical fiber 104 and into a medium surrounding optical fiber 104.

When viewed head-on, the cladding and the core may have various cross-sectional shapes in the non-evanescent portions and the evanescent portions of the optical fiber. In some embodiments, the head-on cross-sectional shape of the cladding in the non-evanescent portion is geometrically similar to the head-on cross-sectional shape of the cladding in the evanescent portion. In other embodiments, the head-on cross-sectional shape of the cladding in the non-evanescent portion is different the head-on cross-sectional shape of the cladding in the evanescent portion. Similarly, in some embodiments, the head-on cross-sectional shape of the core in the non-evanescent portion is geometrically similar to the head-on cross-sectional shape of the core in the evanescent portion. In other embodiments, the head-on cross-sectional shape of the core in the non-evanescent portion is different the head-on cross-sectional shape of the core in the evanescent portion.

A head-on, cross-sectional shape of the cladding in a non-evanescent portion and/or in an evanescent portion of an optical fiber may be circular, triangular, trapezoidal, rectangular, square, annular, pentagonal, hexagonal, heptagonal, octagonal, or elliptical. A head-on, cross-sectional shape of the core in a non-evanescent portion and/or in an evanescent portion of an optical fiber may be circular, triangular, trapezoidal, rectangular, square, annular, pentagonal, hexagonal, heptagonal, octagonal, or elliptical. The head-on, cross-sectional shapes of one or more of the cladding and the core may symmetric or asymmetric about one or more axes in a non-evanescent portion or in an evanescent portion.

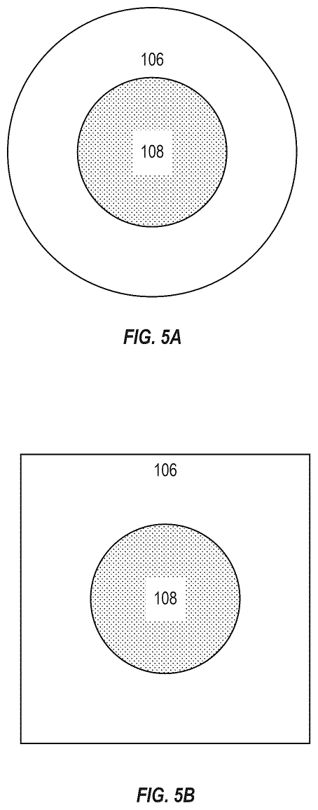

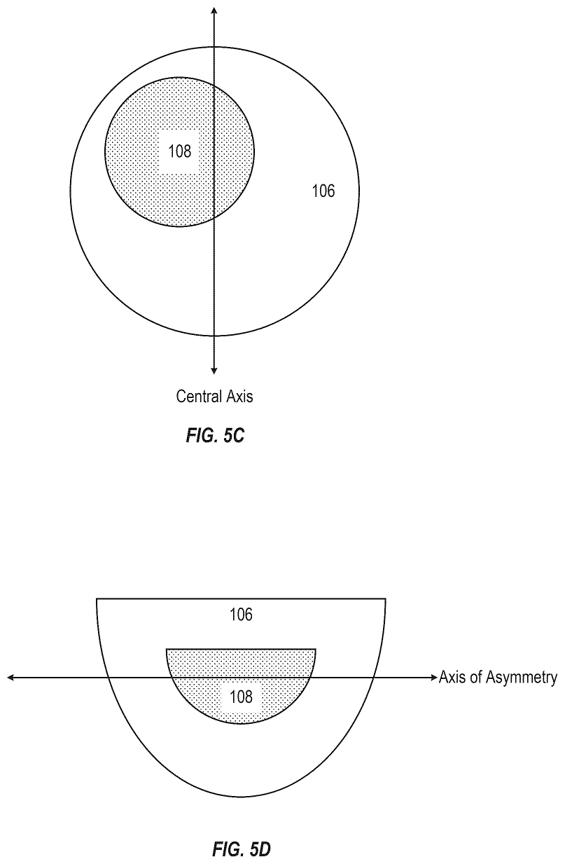

FIGS. 5A-5D show cross-sectional, head-on views of various example optical fibers. Cladding 106 and core 108 may have any of the depicted shapes in any portion (non-evanescent or evanescent) of an optical fiber. For instance, as shown in FIG. 5A, in a non-evanescent portion and/or in an evanescent portion of an optical fiber, core 108 may have a circular cross-section and cladding 106 may have an annular cross section. Alternatively, in a non-evanescent portion and/or in an evanescent portion of an optical fiber, core 108 may have a circular cross-section, while a cross-sectional shape of cladding 106 may be rectangular with an interior circular cutout (FIG. 5B). In some embodiments, in a non-evanescent portion and/or in an evanescent portion of an optical fiber, core 108 is offset from the center of cladding 106 (FIG. 5C). In some embodiments, in a non-evanescent portion and/or in an evanescent portion of an optical fiber, core 108 and/or cladding 106 may be asymmetric about at least one axis (FIG. 5D). Asymmetric evanescent regions may be used to target eccentric lesions.

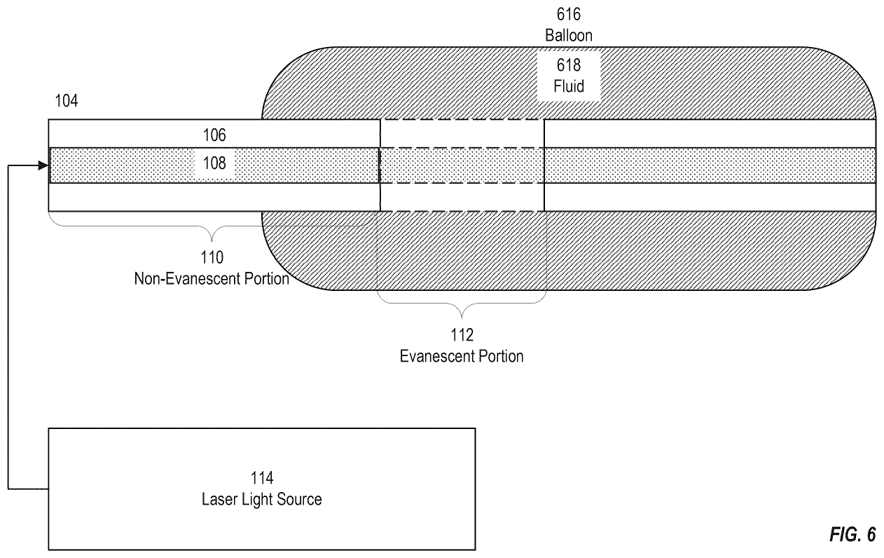

In some embodiments, the provided catheters (e.g., catheter 102 shown in FIG. 1) include a balloon. FIG. 6 shows an example laser lithotripsy system 600 wherein catheter 102 comprises a balloon 616. At least a portion of optical fiber 104 of catheter 102 may be contained within balloon 616. In particular, at least one evanescent portion 112 of optical fiber 104 may be contained with balloon 616. Balloon 616 may also contain a fluid 618 into which evanescent fields from optical fiber 104 may be transmitted. The energy carried by the evanescent fields may heat fluid 618, producing vaporization bubbles. The expansion and subsequent rapid collapse of the vaporization bubbles may create shock waves.

The wall of balloon 616 may comprise a non-porous material, which may facilitate the efficient transfer of energy from the shock waves to treatment sites. In some embodiments, the wall of balloon 616 is formed entirely from a non-porous material. In other embodiments, the wall of balloon 616 is formed predominantly (but not entirely) from a non-porous material. For example. the majority of the wall of balloon 616 may be formed from a non-porous material and a minority portion of the wall of balloon 616 may be formed from a porous material. In other embodiments, the wall of balloon 616 is partially formed from a non-porous material. For instance, half of the surface area of the wall of balloon 616 may be formed from a non-porous material and half of the surface area of the wall of balloon 616 may be formed from a porous material.

Fluid 618 may have a high absorption coefficient for the wavelength of light that is provided to optical fiber 104 by laser light source 114. For example, the absorption coefficient of fluid 618 for light with a wavelength of approximately 2 μm (e.g., 1.99, 2.01, 2.02, 2.03, 2.04, or 2.05 μm) may be approximately 100 cm⁻¹ (e.g., 99.9, 99.99, 100.01, 100.02, or 100.03 cm⁻¹). In various embodiments, fluid 618 may comprise a saline solution or water.

In other embodiments, the provided catheters (e.g., catheter 102 shown in FIG. 1) include an enclosure, and at least a portion of the optical fiber (e.g., optical fiber 104) is contained within the enclosure. For example, at least one evanescent portion of the optical fiber may be contained within the enclosure.

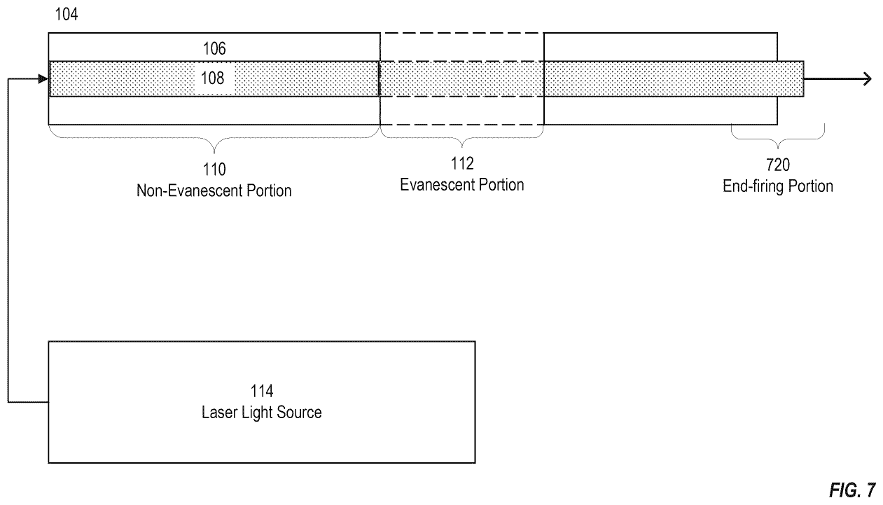

In some embodiments, an optical fiber for a catheter may include an end-firing portion as well as the evanescent and non-evanescent portions described herein. Laser lithotripsy system 700 shown in FIG. 7 includes a catheter 102 with an optical fiber 102 that comprises a distal end-firing portion 720. End-firing portion 102 may be configured to emit light that is not transmitted out of optical fiber 102 as evanescent fields in evanescent portion 112.

The amount of light that is transmitted out of an evanescent portion of an optical fiber may depend upon the optical power of the laser light source that provides the light to the optical fiber as well as the number of evanescent portions that constitute the optical fiber. If an optical fiber comprises multiple evanescent portions, each evanescent portion may emit a fraction of the total amount of light that is provided to the optical fiber by the laser light source.

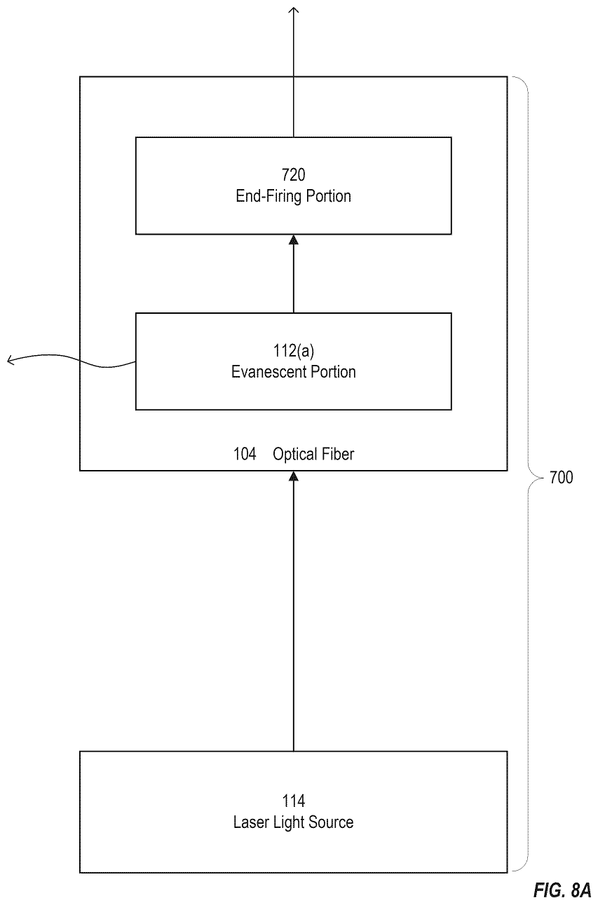

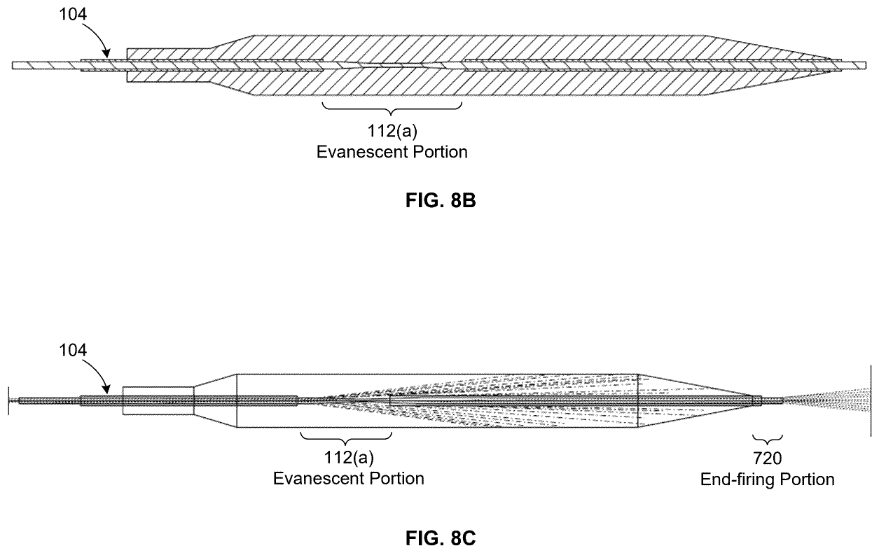

An optical fiber 104 may have a single evanescent portion 112(a), as shown in FIGS. 8A-8C, in which case at least 35%, at least 40%, at least 45%, at least 50%, at least 55%, at least 56%, at least 57%, at least 58%, at least 59%, or at least 60%, of the light that is provided to optical fiber 104 by laser light source 114 may be transmitted out of evanescent portion 112(a) in the form of evanescent fields. End-firing portion 720, if present, may emit about 35%, about 36%, about 37%, about 38%, about 39%, or about 40% of the light that is provided to optical fiber 104 by laser light source 114.

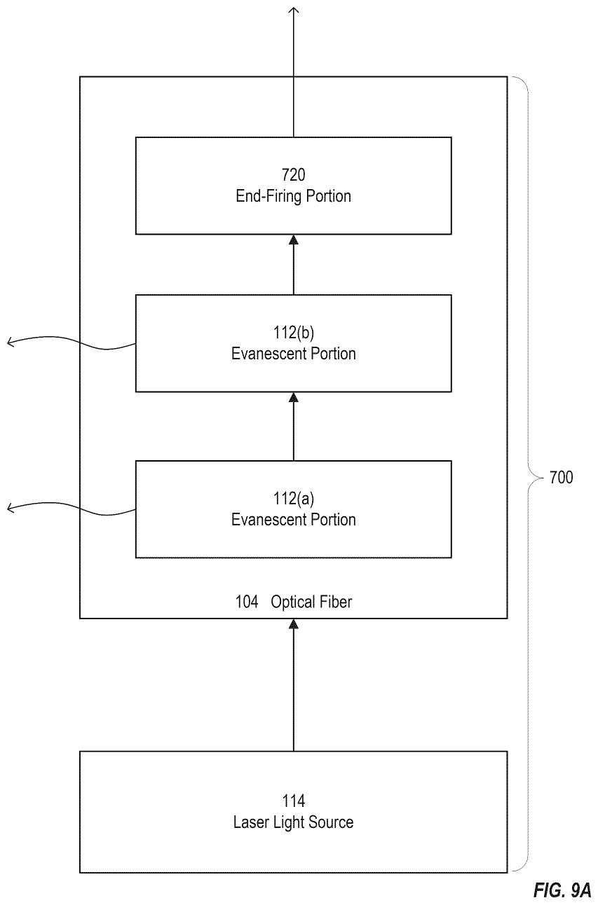

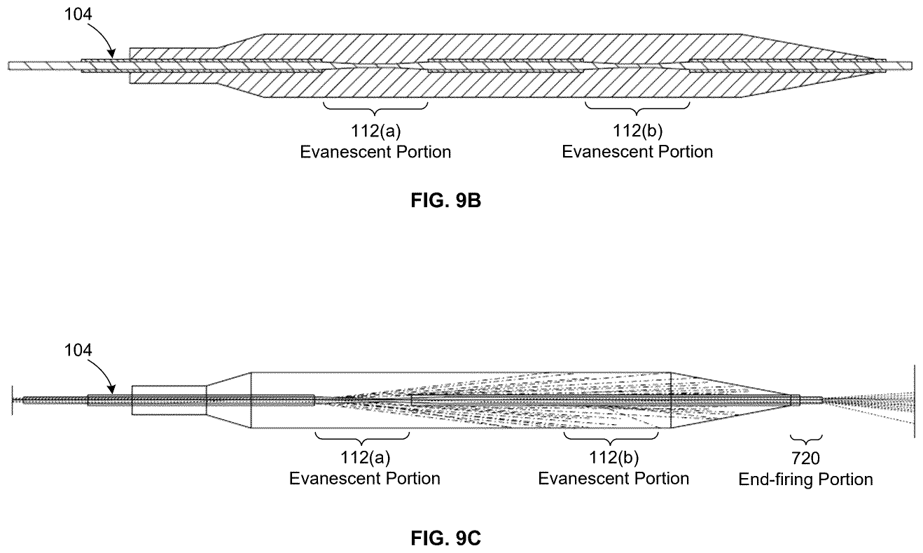

Alternatively, optical fiber 104 may have two evanescent portions 112(a) and 112(b) (FIGS. 9A-9C). The first evanescent portion 112(a) may transmit between 25% and 50%, between 30% and 45%, or between 35% and 40% of the light that is provided to optical fiber 104 by laser light source 114 out of optical fiber 104 as evanescent fields and the second evanescent portion 112(b) may transmit between 20% and 35%, between 20% and 30%, or between 25% and 30% of the light that is provided to optical fiber 104 by laser light source 114 out of optical fiber 104 as evanescent fields. In some embodiments, the first evanescent portion 112(a) transmits about 35%, about 36%, about 37%, about 38%, about 39% or about 40% of the light that is provided to optical fiber 104 by laser light source 114, while the second evanescent portion 112(b) transmits about 25%, about 26%, about 27%, about 28%, about 29%, or about 30% of the light that is provided to optical fiber 104 by laser light source 114. End-firing portion 720, if present, may emit about 30%, about 31%, about 32%, about 33%, about 34%, or about 35% of the light that is provided to optical fiber 104 by laser light source 114.

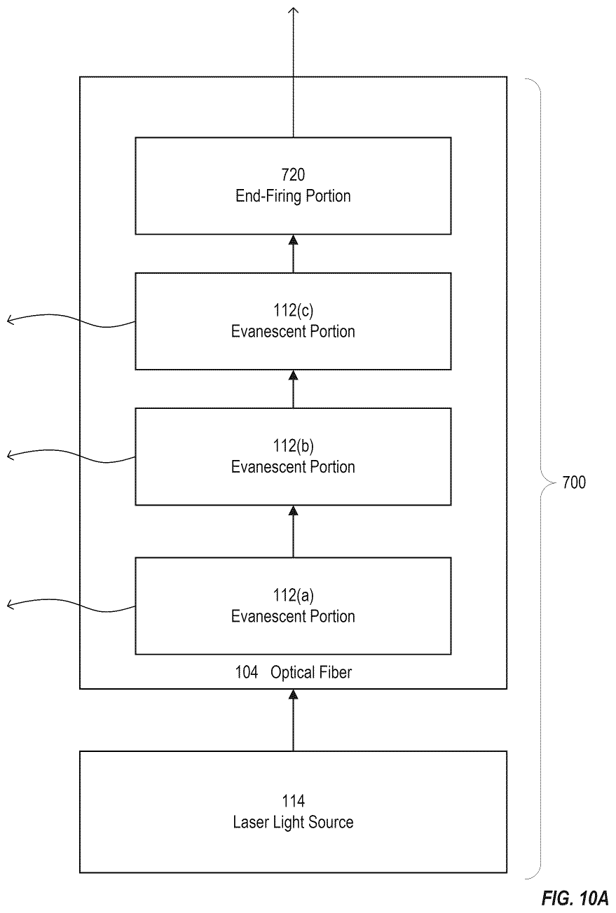

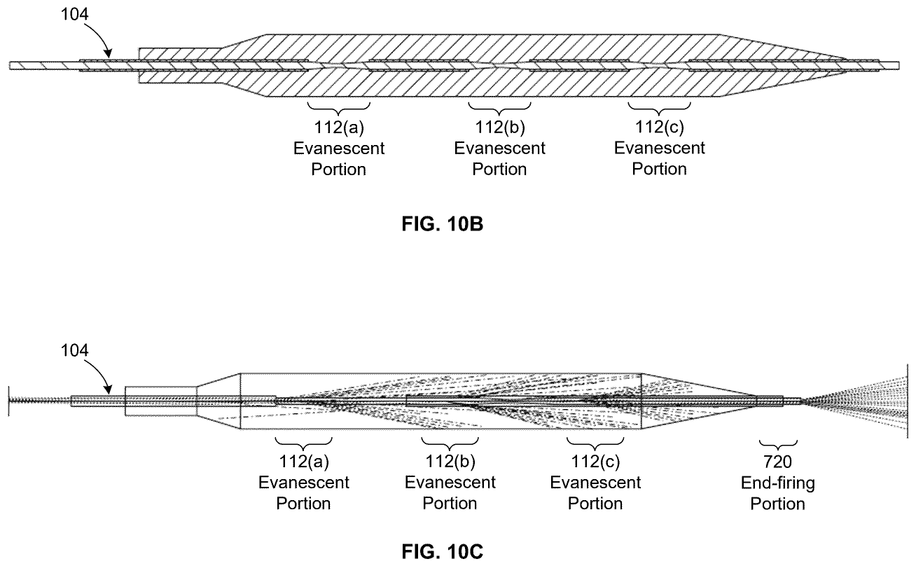

Optical fiber 104 can also have three evanescent portions 102(a), 102(b), and 102(c), as shown in FIGS. 10A-10C. The first evanescent portion 112(a) may transmit between 10% and 60%, between 20% and 50%, or between 30% and 40% of the light that is provided to optical fiber 104 by laser light source 114 out of optical fiber 104 as evanescent fields, for example about 32%, 33%, 34%, 35%, 36%, 37%, or 38% of the light that is provided to optical fiber 104 by laser light source 114. The second evanescent portion 112(b) may transmit between 5% and 30%, between 10% and 25%, or between 15% and 20% of the light that is provided to optical fiber 104 by laser light source 114 out of optical fiber 104 as evanescent fields, for example about 15%, 16%, 17%, 18%, 19%, 20%, or 21% of the light that is provided to optical fiber 104 by laser light source 114. The third evanescent portion 112(c) may transmit between 5% and 30%, between 10% and 25%, or between 15% and 20% of the light that is provided to optical fiber 104 by laser light source 114 out of optical fiber 104 as evanescent fields, for example about 14%, 15%, 16%, 17%, 18%, 19%, or 20% of the light that is provided to optical fiber 104 by laser light source 114. End-firing portion 720, if present, may emit about 24%, about 25%, about 26%, about 27%, about 28%, or about 29% of the light that is provided to optical fiber 104 by laser light source 114.

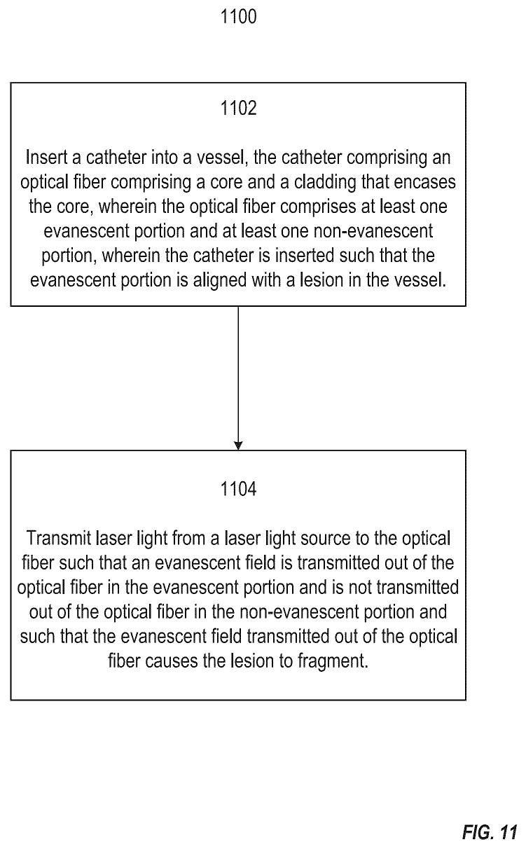

A method 1100 of using a laser lithotripsy system such as system 100 (FIG. 1), system 600 (FIG. 6), or system 700 (FIG. 7) is provided in FIG. 11. As shown, in a first step 1102, a catheter such as one of the catheters provided herein (e.g., catheter 102 shown in FIG. 1) may be inserted into a vessel or other bodily structure. The catheter may be inserted using any suitable technique. Furthermore, the catheter may be inserted so that an evanescent portion (e.g., evanescent portion 112) of the optical fiber (e.g., optical fiber 104) is aligned with a lesion in the vessel. After the catheter has been appropriately inserted, light from a laser light source (e.g., laser light source 114) may be transmitted into the optical fiber. As this light propagates through the optical fiber, evanescent fields may be transmitted out of the optical fiber through the evanescent portion(s) of the optical fiber. The transmitted evanescent fields may produce shock waves (e.g., by heating a fluid that surrounds the optical fiber) that propagate to the lesion, thereby causing the lesion to fragment (step 1104).

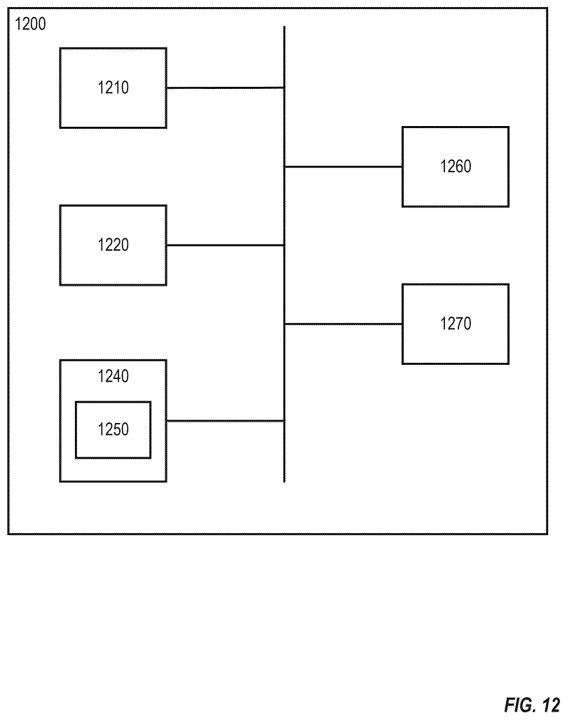

In various embodiments, a computer system can be used in combination with the catheter devices described herein, for example to tune or otherwise control laser parameters (e.g., laser frequency, pulse frequency, intensity, etc.), to receive and process data associated with the catheter devices or a lesion for which a catheter device is being used (e.g., imaging sensor data, pressure sensor data, etc.), to interface with one or more control devices and/or graphical user interfaces usable to control the system(s) described herein, and/or to interface with one or more network/connected devices to send and/or receive data (e.g., control data, data generated by monitoring system usage, sensor data, etc.). An exemplary computer system 1200 is provided in FIG. 12. System 1200 can be any suitable type of processor-based system, such as a personal computer, workstation, server, handheld computing device (portable electronic device) such as a phone or tablet, or dedicated device. The system 1200 can include, for example, one or more of input device 1220, output device 1230, one or more processors 1210, storage 1240, and communication device 1260. Input device 1220 and output device 1230 can generally correspond to those described above and can either be connectable or integrated with the computer.

Input device 1220 can be any suitable device that provides input, such as a push-button switch, a touch screen, keyboard or keypad, mouse, gesture recognition component of a virtual/augmented reality system, or voice-recognition device. Output device 1230 can be or include any suitable device that provides output, such as a display, touch screen, haptics device, virtual/augmented reality display, or speaker.

Storage 1240 can be any suitable device that provides storage, such as an electrical, magnetic, or optical memory including a RAM, cache, hard drive, removable storage disk, or other non-transitory computer readable medium. Communication device 1260 can include any suitable device capable of transmitting and receiving signals over a network, such as a network interface chip or device. The components of the computing system 1200 can be connected in any suitable manner, such as via a physical bus or wirelessly.

Processor(s) 1210 can be any suitable processor or combination of processors, including any of, or any combination of, a central processing unit (CPU), graphics processing unit (GPU), field programmable gate array (FPGA), programmable system on chip (PSOC), and application-specific integrated circuit (ASIC). Software 1250, which can be stored in storage 1240 and executed by one or more processors 1210, can include, for example, the programming that embodies the functionality or portions of the functionality of the present disclosure (e.g., as embodied in the devices as described above)

Software 1250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1240, that can contain or store programming for use by or in connection with an instruction execution system, apparatus, or device.

Software 1250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport computer readable medium can include, but is not limited to, an electronic, magnetic, optical, electromagnetic, or infrared wired or wireless propagation medium.

System 1200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

System 1200 can implement any operating system suitable for operating on the network. Software 1250 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

The catheter devices described herein can be used for a variety of occlusions, including coronary occlusions, such as lesions in vasculature, or occlusions in the peripheral vasculature (e.g., above-the-knee, below-the-knee, iliac, carotid, etc.). For further examples, similar designs may be used for treating soft tissues, such as cancer and tumors (i.e., non-thermal ablation methods), blood clots, fibroids, cysts, organs, scar and fibrotic tissue removal, or other tissue destruction and removal. Catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

The elements and features of the exemplary catheters discussed above may be rearranged, recombined, and modified, without departing from the present invention. Furthermore, numerical designators such as “first”, “second”, “third”, “fourth”, etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It should be noted that the elements and features of the example catheters illustrated throughout this specification and drawings may be rearranged, recombined, and modified without departing from the present invention. For instance, the present disclosure is intended to include catheters having a variety of balloon configurations. The number, placement, and spacing of the evanescent regions can be modified without departing from the subject invention. Further, the number, placement, and spacing of balloons of catheters can be modified without departing from the subject invention.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A laser lithotripsy system comprising:
a light energy source; and
a catheter including an optical fiber at least partially contained with a balloon of the catheter, the optical fiber configured to be optically coupled to receive laser light from the light energy source, and the optical fiber comprising:
a lengthwise portion including a first evanescent portion contained within the balloon and a first non-evanescent portion contained within the balloon;
a core;
a cladding that fully encases the core along the lengthwise portion, wherein the laser light propagating through the first evanescent portion is transmitted out of the optical fiber via an evanescent field and into the balloon, and wherein the laser light propagating through the first non-evanescent portion is not transmitted out of the optical fiber nor into the balloon; and
a distal end located outside of the balloon, the distal end configured to emit the laser light that propagates through the first evanescent portion.

2. The laser lithotripsy system of claim 1, wherein the core has a first radius in the first non-evanescent portion and a second radius less than the first radius in the first evanescent portion.

3. The laser lithotripsy system of claim 2, wherein the first radius is greater than or equal to 325 μm and less than or equal to 375 μm.

4. The laser lithotripsy system of claim 2, wherein the second radius is greater than or equal to 50 μm and less than or equal to 350 μm.

5. The laser lithotripsy system of claim 2, wherein, starting at an interface between the first non-evanescent portion and the first evanescent portion, a core radius tapers from the first radius to the second radius.

6. The laser lithotripsy system of claim 1, further comprising a second non-evanescent portion, wherein:
the first evanescent portion is between the first and second non-evanescent portions;
a core radius tapers from an interface between the first non-evanescent portion and the first evanescent portion toward a central region of the first evanescent portion; and
the core radius expands from the central region toward an interface between the first evanescent portion and the second non-evanescent portion.

7. The laser lithotripsy system of claim 5, wherein a taper angle of the core is greater than or equal to 0.5° and less than or equal to 5°.

8. The laser lithotripsy system of claim 1, wherein the cladding has a first outer radius in the first non-evanescent portion and a second outer radius less than the first outer radius in the first evanescent portion.

9. The laser lithotripsy system of claim 8, wherein the first outer radius is greater than or equal to 375 μm and less than or equal to 425 μm.

10. The laser lithotripsy system of claim 8, wherein the second outer radius is greater than or equal to 50 μm and less than or equal to 375 μm.

11. The laser lithotripsy system of claim 8, wherein, starting at an interface between the first non-evanescent portion and the first evanescent portion, a cladding outer radius tapers from the first outer radius to the second outer radius.

12. The laser lithotripsy system of claim 1, further comprising a second non-evanescent portion, wherein:
the first evanescent portion is between the first and second non-evanescent portions;
a cladding outer radius tapers from an interface between the first non-evanescent portion and the first evanescent portion toward a central region of the first evanescent portion; and
the cladding outer radius expands from the central region toward an interface between the first evanescent portion and the second non-evanescent portion.

13. The laser lithotripsy system of claim 11, wherein a taper angle of the cladding is greater than or equal to 0.5° and less than or equal to 5°.

14. The laser lithotripsy system of claim 11, wherein the cladding outer radius tapers continuously from the first outer radius to the second outer radius.

15. The laser lithotripsy system of claim 11, wherein the cladding outer radius from the first outer radius to the second outer radius is variable.

16. The laser lithotripsy system of claim 1, wherein a ratio of a cladding outer radius in the first non-evanescent portion to the cladding outer radius in the first evanescent portion is greater than 1 and less than or equal to 6.5.

17. The laser lithotripsy system of claim 1, wherein a ratio of a core radius in the first non-evanescent portion to the core radius in the first evanescent portion is greater than 1 and less than or equal to 6.5.

18. The laser lithotripsy system of claim 1, wherein a ratio of a cladding outer radius to a core radius in the first non-evanescent portion is between 0.5 and 0.9.

19. The laser lithotripsy system of claim 1, wherein a ratio of a cladding outer radius to a core radius in the first evanescent portion is between 0.025 and 0.875.

20. The laser lithotripsy system of claim 1, wherein the cladding is between 10% and 50% thinner in the first evanescent portion than in the first non-evanescent portion.

21. The laser lithotripsy system of claim 1, wherein a cross-sectional shape of the cladding in the first evanescent portion is asymmetrical.

22. The laser lithotripsy system of claim 1, wherein a cross-sectional shape of the core in the first evanescent portion is asymmetrical.

23. The laser lithotripsy system of claim 1, wherein at least 55% of the laser light received by the optical fiber is transmitted out of the first evanescent portion.

24. The laser lithotripsy system of claim 1, wherein the optical fiber comprises a second evanescent portion, wherein:
greater than or equal to 35% and less than or equal to 40% of the laser light received by the optical fiber is transmitted out of the first evanescent portion; and
greater than or equal to 25% and less than or equal to 30% of the laser light received by the optical fiber is transmitted out of the second evanescent portion.

25. The laser lithotripsy system of claim 1, wherein the optical fiber comprises a second evanescent portion and a third evanescent portion, wherein:
greater than or equal to 30% and less than or equal to 40% of the laser light received by the optical fiber is transmitted out of the first evanescent portion;
greater than or equal to 15% and less than or equal to 20% of the laser light received by the optical fiber is transmitted out of the second evanescent portion; and
greater than or equal to 15% and less than or equal to 20% of the laser light received by the optical fiber is transmitted out of the third evanescent portion.

26. (canceled)

27. The laser lithotripsy system of claim 1, wherein greater than or equal to 25% and less than or equal to 40% of the laser light received by the optical fiber is emitted at the distal end of the optical fiber.

28. (canceled)

29. The laser lithotripsy system of claim 1, wherein the balloon is configured to contain a fluid, wherein, for a wavelength of the light energy source, the fluid has an absorption coefficient of at least 100 cm⁻¹.

30. The laser lithotripsy system of claim 28, wherein the fluid is an aqueous fluid.

31. The laser lithotripsy system of claim 1, wherein the catheter comprises an enclosure, wherein the optical fiber is contained within the enclosure.

32. The laser lithotripsy system of claim 1, wherein the light energy source is a laser light source.

33. The laser lithotripsy system of claim 1, wherein a wavelength of the light energy source is between 1 μm and 3 μm.

34. The laser lithotripsy system of claim 1, wherein, for a wavelength of the light energy source, an index of refraction of the core is greater than an index of refraction of the cladding.

35. The laser lithotripsy system of claim 1, wherein, for a wavelength of the light energy source, an index of refraction of the core is between 1.43 and 1.44.

36. The laser lithotripsy system of claim 1, wherein, for a wavelength of the light energy source, an index of refraction of the cladding is between 1.4 and 1.42.

37. The laser lithotripsy system of claim 1, wherein an optical power density of light emitted by the light energy source is between 0.01 W/cm² and 1×10¹¹ W/cm².

38. The laser lithotripsy system of claim 1, wherein:
in the first evanescent portion, the evanescent field penetrates into the cladding, decays exponentially as a function of distance from the core within the cladding, and is transmitted out of the optical fiber; and
in the first non-evanescent portion, the evanescent field penetrates into the cladding, decays exponentially as a function of distance from the core within the cladding, and is not transmitted out of the optical fiber.

39. The method of claim 1, wherein the laser light emitted from the distal end is based at least in part on the laser light transmitted out of the optical fiber via the evanescent field.

40. The method of claim 1, wherein the laser light emitted from the distal end decreases as a number of evanescent portions included in the lengthwise portion increases.
